(19) United States
(12) Patent Application Publication
(10) Pub. No.: US 2004/0225128 A1
(43) Pub. Date: Nov. 11, 2004

(54) Paroxetine isethionate salt, process of preparation and use in the treatment of depression

(76) Inventor: George Leo Callewaert, Buckinghamshire (GB)

Correspondence Address: OPPEDAHL AND LARSON LLP, P O BOX 5068, DILLON, CO, 80435-5068, US

(21) Appl. No.: 10/485,705

(22) PCT Filed: Jul. 24, 2002
(86) PCT No.: PCT/GB02/03377

(30) Foreign Application Priority Data
Aug. 2, 2001 (GB) 0118869.7
Nov. 9, 2001 (GB) 0127203.8

(51) Int. Cl. C 07D 4 5/02

(52) U.S. Cl. 546/197

(57) ABSTRACT

A salt derived from isethionic acid and paroxetine free base of formula (I)

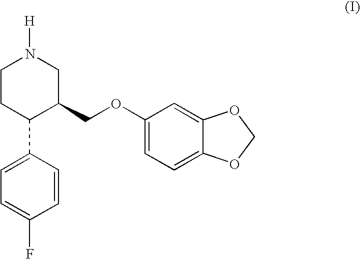

This application is a Section 371 national phase of PCT/GB02/03377, filed Jul. 24, 2002 designating the United States and published in English on Mar. 13, 2003.

### BACKGROUND OF THE INVENTION

The present invention is concerned with a salt of paroxetine, its preparation and its use as a therapeutic agent or an intermediate.

U.S. Pat. No. 4,007,196 discloses a piperidine derivative of formula I, which is (−)-trans-4-(4′-fluorophenyl)-3-(3′4′-methylenedioxyphenoxymethyl)-piperidine

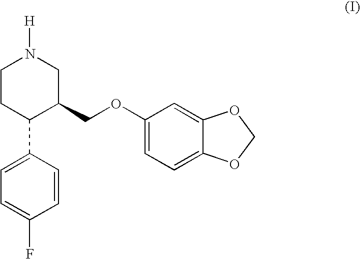

The above compound, known by the generic name, paroxetine, is an inhibitor of 5-hydroxytryptamine (5HT) uptake and is useful for the treatment of depressive illness and other related illnesses. U.S. Pat. No. 4,007,196 also describes the preparation of paroxetine as both the free base and the maleate salt.

European Patent 0223403B discloses the preparation of the hydrochloride salt of paroxetine, both as the anhydrate and as the hemihydrate. The hemihydrate salt is described as being crystalline, stable and non-hygroscopic. Furthermore, because of its more favourable handling characteristics, the hemihydrate salt is more suitable for commercial use. European Patent 0223403B also discloses a method for the preparation of the crystalline acetate salt, which is useful as an intermediate for the preparation of the hydrochloride salt.

PCT Patent Application WO 98/56787 discloses sulphonic acid salts of paroxetine.

PCT Patent Application WO 99/40084 discloses a number of further salts of paroxetine and pharmaceutically acceptable acids.

PCT Patent Application WO 00/01692 discloses a very wide range of salts of paroxetine, the salts being derived from a wide range of organic acids and paroxetine.

Many of the paroxetine salts disclosed in the above prior art patent documents exist only as oils and, therefore, are unsuitable for general pharmaceutical use. Other prior art paroxetine salts which are crystalline, exist in the form of poorly flowing powders or are hygroscopic, and are, therefore, also unsuitable for pharmaceutical use.

### SUMMARY OF THE INVENTION

There is, therefore, a need for a paroxetine salt with improved pharmaceutical characteristics, and such a paroxetine salt is provided by the present invention, which paroxetine salt according to the present invention alleviates the hitherto problems associated with prior art paroxetine salts as described above.

More particularly, there is provided by the present invention a salt derived from isethionic acid (2-hydroxyethyl sulphonic acid) and paroxetine free base of formula (I)

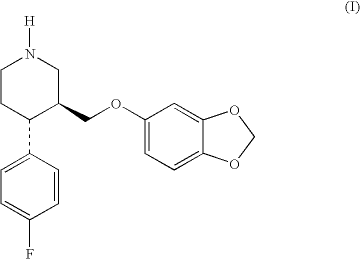

For example, there is provided by the present invention paroxetine isethionate as represented by formula (II)

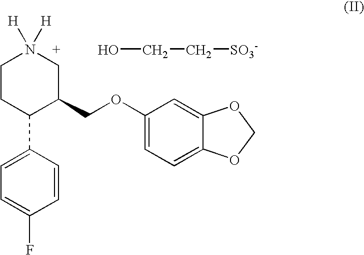

### BRIEF DESCRIPTION OF THE FIGURES

FIG. 1 shows an infrared spectrum of paroxetine isethionate from Example 3.

FIG. 2 shows an X-ray powder diffractogram of paroxetine isethionate from Example 3.

FIGS. 3(a) and (b) shows a differential calorimetry scan of paroxetine isethionate from Example 3.

FIG. 4 shows an infrared spectrum of paroxetine isethionate from Example 4.

FIG. 5 shows an X-ray powder diffractogram of paroxetine isethionate from Example 4.

FIG. 6 shows a differential calorimetry scan of paroxetine isethionate from Example 4.

FIG. 7 shows an infrared spectrum of paroxetine isethionate from Example 5.

FIG. 8 shows an X-ray powder diffractogram of paroxetine isethionate from Example 5.

FIG. 9 shows a differential calorimetry scan of paroxetine isethionate from Example 5.

### DETAILED DESCRIPTION OF THE INVENTION

Surprisingly, it has now been found that the above mentioned isethionate salt of paroxetine of formula (II) according to the present invention alleviates the hitherto problems associated with prior art paroxetine salts as described above. More particularly, paroxetine isethionate of formula (II) according to the present invention is preferably provided in crystalline form and may exist in the form of small crystalline grains, which can be provided as a free flowing powder, thus exhibiting desirable, improved pharmaceutical properties.

A paroxetine salt according to the present invention may be prepared by methods known per se. For example, a paroxetine salt according to the present invention may be produced by reacting paroxetine free base of formula (I) or a salt thereof and isethionic acid (or a salt thereof) in a suitable solvent and isolating the resulting salt, provided seed crystals are present in the environment.

A paroxetine isethionate salt according to the present invention may alternatively be prepared by chemical modification of a precursor isethionate salt. Suitable precursors are those which may be converted to a paroxetine isethionate salt according to the present invention by hydrogenation or the like. For example, the N-benzyl derivative of paroxetine isethionate in a suitable solvent (such as a C₁-4alkanol or the like) may be hydrogenated using a suitable catalyst, such as palladium on charcoal, to generate a solution of a paroxetine isethionate salt according to the present invention.

Derivatives of paroxetine which react with isethionic acid to form a paroxetine isethionate salt according to the present invention may also be used. Examples of such derivatives include salts of paroxetine with acids, the pKa of which is higher than the pKa of isethionic acid and examples of such acids are weak organic acids, such as the carboxylic acids. A further suitable paroxetine derivative includes paroxetine N protected with an acid labile group and examples of such acid labile groups are trimethyl-silyl, tertiary butyloxycarbonyl and the like.

Isolation of a paroxetine salt according to the present invention may include concentration or precipitation, for example by addition of an additional solvent which is miscible with the first solvent and in which a paroxetine salt according to the present invention is substantially insoluble. Alternatively, if it is desired to form a paroxetine salt according to the present invention in an organic solvent, it may be beneficial to select an organic solvent in which the paroxetine salt is less soluble than is paroxetine free base. Furthermore, as described above salts of isethionic acid may be used in place of isethionic acid, for example the ammonium salt of isethionic acid or the like.

As is well known in the pharmaceutical field, for many crystalline materials once seed crystals become present in the environment, supersaturated solutions will crystallise spontaneously. This has been found to be true for a paroxetine isethionate salt according to the present invention. Therefore, suitable precautions such as careful temperature control need to be taken when reproducible crystallisations of a paroxetine isethionate salt according to the present invention are needed. Similarly, otherwise difficult or otherwise impossible to crystallise preparations, or crystallisation mixtures in which there are impurities, form crystals once seed crystals are present in the environment.

A wide range of solvents is suitable for use in the formation of a paroxetine salt according to the present invention, but those solvents which promote ionisation and hence salt formation are particularly preferred. Examples of such suitable solvents include one or more of water,

C₁-C₆ alkanols, C₁-C₆ esters, C₁-C₆ ketones, halogenated hydrocarbons, C₁-C₆ ethers, acetonitrile, dimethylformamide and the like. As will be appreciated from the above, mixtures of such solvents may be used. Once formed, paroxetine isethionate may be dissolved in and crystallised from other solvents, such as toluene or the like.

A paroxetine isethionate salt according to the present invention may, according to its precise method of formation and isolation, contain variable amounts of water or solvent. The hydrates and solvates of paroxetine isethionate of formula (II) are, therefore, to be considered as falling within the scope of the present invention.

A paroxetine isethionate salt according to the present invention, prepared substantially as herein described, exists as a single polymorph as can be seen by reference to X-ray diffraction analysis and differential scanning calorimetry carried out according to Examples 3, 4 and 5. Other polymorphs of a paroxetine isethionate salt according to the present invention may, however, be derived from alternative crystallisation conditions and all such polymorphs are to be considered as falling within the scope of the present invention.

A paroxetine isethionate salt according to the present invention may be used as a medicament, particularly in the treatment of depression or other disease states where the use of a 5HT uptake inhibitor is indicated. More particularly, a paroxetine isethionate salt according to the present invention can be used to treat any of the following disorders where the uptake of a 5HT uptake inhibitor can be beneficial—alcoholism, depression (for example adolescent depression), panic disorder, obesity, migraine, anorexia, pre-menstrual syndrome, trichotillomania, substance abuse, anxiety, obsessive compulsive disorder, chronic pain, senile dementia, bulimia, social phobia, dysthymia and the like. A paroxetine isethionate salt according to the present invention is especially useful for the treatment of depression, obsessive compulsive disorder, panic and the like.

There is provided by the present invention, therefore, for use in therapy a salt derived from paroxetine free base of formula (I) and isethionic acid, in particular paroxetine isethionate of formula (II). Paroxetine isethionate of formula (II) according to the present invention is pharmaceutically acceptable and may be used in the preparation of tablets, capsules and other pharmaceutical presentations.

The present invention also provides a pharmaceutical composition comprising a salt derived from paroxetine free base of formula (I) and isethionic acid, in particular paroxetine isethionate of formula (II), together with a pharmaceutically acceptable carrier, diluent or excipient therefor.

The compositions according to the present invention are usually adapted for oral administration, but compositions for dissolution for parenteral administration are also within the scope of the present invention. More particularly, a paroxetine isethionate salt according to the present invention is extremely soluble in water and, therefore, is well suited for use in a liquid pharmaceutical composition. Surprisingly, however, a paroxetine isethionate salt according to the present invention (unlike many other isethionate salts with amines) exhibits practically no adverse hygroscopicity characteristics. Also, surprisingly paroxetine isethionate has superior taste characteristicss.

Compositions according to the present invention are usually presented as unit dose compositions containing from 1 to 200 mg, more usually from 5 to 100 mg, for example 10 to 50 mg such as 12.5, 15, 20, 25, 30 or 40 mg of active ingredient. Such compositions are normally taken from 1 to 6 times daily, for example 2, 3 or 4 times daily so that the total amount of active agent administered is within the range of 5 to 400 mg.

In the literature there are numerous examples of pharmaceutical formulations which include paroxetine, for example such as those described in U.S. Pat. No. 6,113,944, U.S. Pat. No. 6,300,343, US 20020032220, WO 02/17921, EP 0734260, US 20020028242, US 20020086053 and US 20020090394.

Suitably, a paroxetine isethionate salt according to the present invention may be formulated for administration by any route, and examples are oral, rectal, topical, parenteral, intravenous or intramuscular administration. Preparations may, if desired, be designed to give slow release of a paroxetine isethionate salt according to the present invention.

The medicaments may, for example, be in the form of tablets, capsules, sachets, vials, powders, granules, lozenges, reconstitutable powders, or liquid preparations, for example solutions or suspensions, or suppositories.

The medicaments, for example those suitable for oral administration, may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycerine; tabletting lubricants, for example magnesium stearate; disintegrants, for example starch, polyvinylpyrrolidone, sodium starch glycollate or microcrystalline cellulose; or pharmaceutically acceptable setting agents such as sodium lauryl sulphate.

Solid medicaments may be obtained by conventional methods of blending, filling, tabletting or the like. Repeated blending operations may be used to distribute paroxetine isethionate throughout those medicaments employing large quantities of fillers. When the medicament is in the form of a tablet, powder, or lozenge, any carrier suitable for formulating solid pharmaceutical compositions may be used, examples being magnesium stearate, starch, glucose, lactose, sucrose, rice flour and chalk. Tablets may be coated according to methods well known in normal pharmaceutical practice, in particular with an enteric coating. The medicament may also be in the form of an ingestible capsule, for example of gelatin containing paroxetine or a salt thereof if desired with a carrier or other excipients.

Medicaments for oral administration as liquids may be in the form of, for example, emulsions, syrups, or elixirs, or may be presented as a dry product for reconstitution with water or other suitable vehicle before use. Such liquid medicaments may contain conventional additives such as suspending agents, for example sorbitol, syrup, methyl cellulose, gelatin, hydroxyethylcellulose, carboxymethylcellulose, aluminium stearate gel, hydrogenated edible fats; emulsifying agents, for example lecithin sorbitan monooleate, or acacia; aqueous or non-aqueous vehicles, which include edible oils, for example almond oil, fractionated coconut oil, oily esters, for example water or normal saline; preservatives, for example methyl or propyl p-hydroxybenzoate or sorbic acid; and if desired conventional flavouring or colouring agents.

A paroxetine isothionate salt according to the present invention may also be administered by a non-oral route. In accordance with routine pharmaceutical procedure, the medicaments may be formulated, for example for rectal administration as a suppository they may also be formulated for presentation in an injectable form in an aqueous or non-aqueous solution, suspension or emulsion in a pharmaceutically acceptable oil or a mixture of liquids. The liquid may contain bacteriostatic agents, anti-oxidants or other preservatives, buffers or solutes to render the solution isotonic with the blood, thickening agents, suspending agents or other pharmaceutically acceptable additives. Such forms will be presented in unit dose form such as ampoules or disposable injection devices or in multi-dose forms such as bottle from which the appropriate dose may be withdrawn or a solid form or concentrate which can be used to prepare an injectable formulation.

The present invention also provides a method of treatment of an animal patient suffering from, or susceptible to, a disease state ameliorated by the administration of a 5HT uptake inhibitor (in particular a method of treating depression), which method comprises administering an effective amount of a salt derived from paroxetine free base of formula (I) and isethionic acid, in particular paroxetine isethionate of formula (II).

The present invention further provides a salt derived from paroxetine free base of formula (I) and isethionic acid, in particular paroxetine isethionate of formula (II), for use in the treatment of a disease state ameliorated by the administration of a 5HT uptake inhibitor, in particular depression, and there is still further provided by the present invention use of a salt derived from paroxetine free base of formula (I) and isethionic acid, in particular paroxetine isethionate of formula (II), in the manufacture of a medicament for the treatment of a disease state ameliorated by the administration of a 5HT uptake inhibitor, in particular depression.

In addition, paroxetine isethionate according to the present invention may be used as an intermediate in the preparation of other salts of paroxetine and there is, therefore, further provided by the present invention use of a salt derived from paroxetine free base of formula (I) and isethionic acid, in particular paroxetine isethionate of formula (II), as an intermediate in the preparation of a further pharmaceutically acceptable salt of paroxetine. The present invention also provides a process of preparing a further pharmaceutically acceptable salt of paroxetine, which process comprises converting a paroxetine isethionate salt according to the present invention into the further pharmaceutically acceptable salt.

The present invention will be further illustrated by the following Examples which do not limit the scope of the invention in any way.

### EXAMPLES

### Example 1

Paroxetine free base (0.5 gram) was taken up in isopropanol (5.0 ml). Ammonium isethionate (0.23 gram) was then added and the mixture heated with swirling to the point where the isopropanol just began to boil. Over a period of 5-10 minutes the ammonium isethionate gradually dissolved and ammonia gas was given off. Isopropanol was then removed by evaporation until the volume was about 2.0 ml and the solution cooled to room temperature. A few drops of this solution were transferred to each of a series of test tubes. With shaking a second solvent was added to the point where oily drops formed or, if oily drops did not form to a volume of approximately 1 ml. The following solvents were added as second solvent:

Toluene

Heptane

Cyclohexane

Dietheyl ether

Diisopropyl ether

Ethyl acetate

Acetone

Acetonitrile

Chloroform

Dimethoxymethane

Tetrahydrofuran

2-ethyl hexanol

The sealed tubes were left to stand at ambient temperature.

After six weeks crystals were observed to have formed in the tube containing diethyl ether as second solvent.

After three months similar crystals were observed to have formed in the tube containing diisopropyl ether as second solvent. Other tubes showed no crystal formation—only gummy deposits or unchanged solutions.

A further sample of paroxetine free base (0.5 gram) was treated in an identical fashion to give 2.0 ml of a solution of paroxetine isethionate in isopropanol. Addition of a small sample of the crystals caused a rapid crystallisation of paroxetine isethionate to occur. The resulting thick crystalline mass was mobilised by stirring with diethyl ether 10 ml. The crystals were filtered, washed with more ether and dried in vacuo.

### Example 2

Paroxetine free base (1.0 gram) was taken up in isopropanol (10.0 ml). Ammonium isethionate (0.45 gram) was then added and the mixture stirred and heated to reflux. The ammonium isethionate gradually dissolved and ammonia gas was given off. When evolution of ammonia had ceased the solution was cooled and the isopropanol removed by evaporation under reduced pressure to leave a viscous oil. Trituration of this oil with a little ethyl acetate caused rapid formation of crystalline paroxetine isethionate in the form of small crystals. This product was removed by filtration, washed with a small amount of ethyl acetate and dried in vacuo to give a white free flowing powder.

### Example 3

Paroxetine free base (1.0 gram) was taken up in isopropanol (10.0 ml). Ammonium isethionate (0.45 gram) was then added and the mixture stirred and heated to reflux. The ammonium isethionate gradually dissolved and ammonia gas was given off. When evolution of ammonia had ceased the solution was cooled and a small sample of the crystals obtained in Example 1 above were added with stirring. Crystalline paroxetine isethionate formed as needles. With continued stirring the mixture was cooled to 5C. The white crystalline product was filtered, washed successively with a little isopropanol and diethyl ether and dried in vacuo.

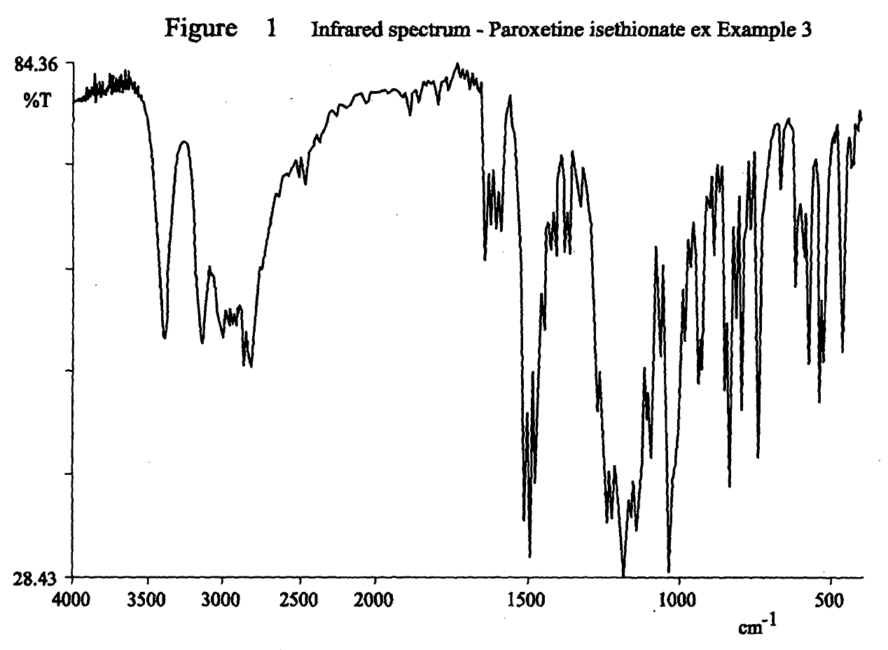

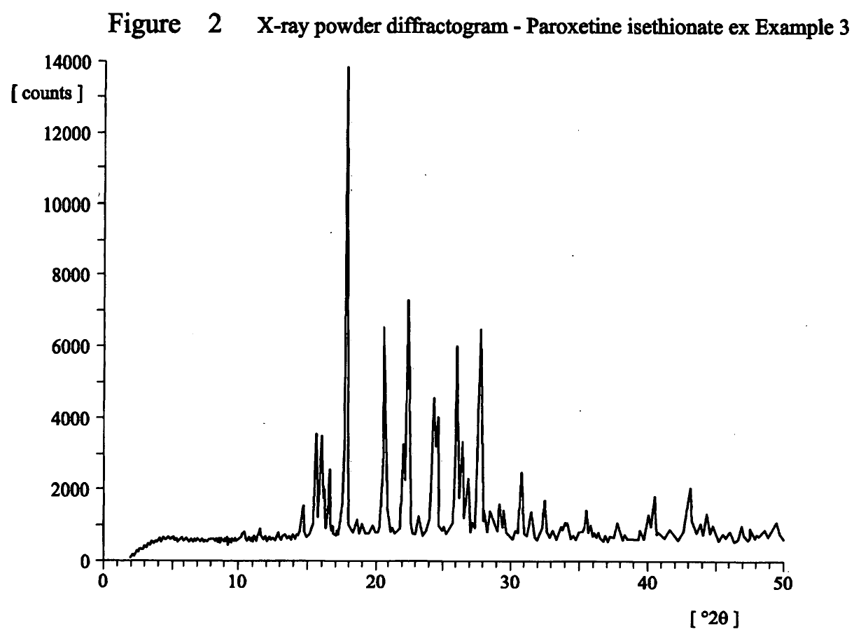

Analytical investigation of this product gave the following results:

| (i) | Elemental analysis: | | | |
|---|---|---|---|---|
| | % m/m | C | H | N |
| | Sample | 55.3 | 5.70 | 3.03 |
| | C₂₁H₂₆FNO₇S requires | 55.4 | 5.75 | 3.07 |

| | | |
|---|---|---|
| (ii) | Melting point: | 126.5° C. |
| (iii) | Infrared spectrum: | See FIG. 1 |
| (iv) | X-ray powder diffractogram: | See FIG. 2 |

Table 1 gives the conditions used to obtain this diffractogram and a listing of peak positions and intensities.

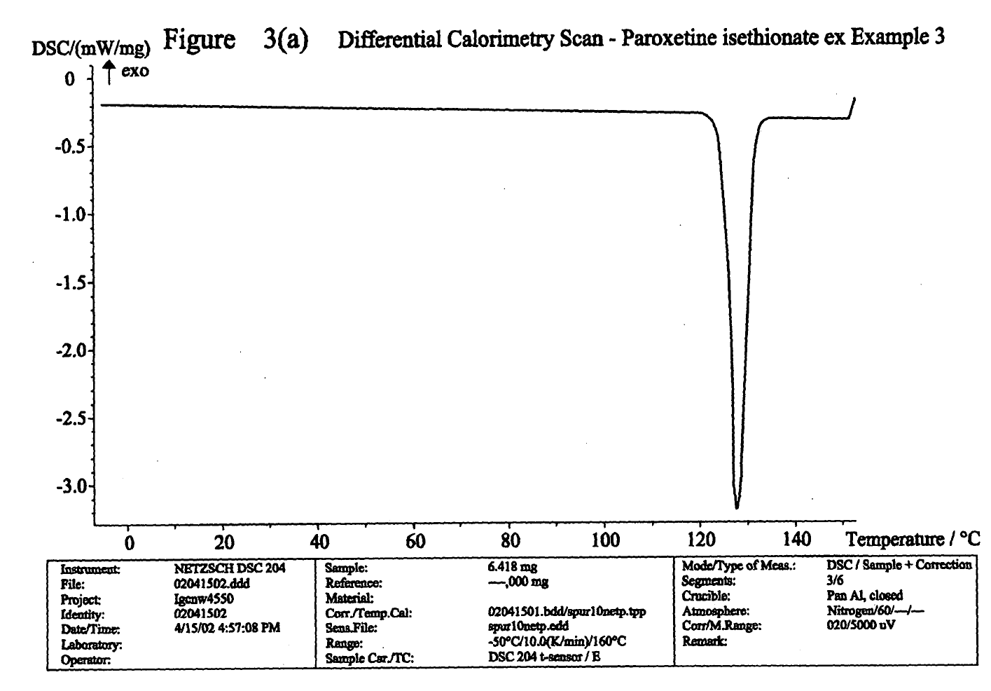

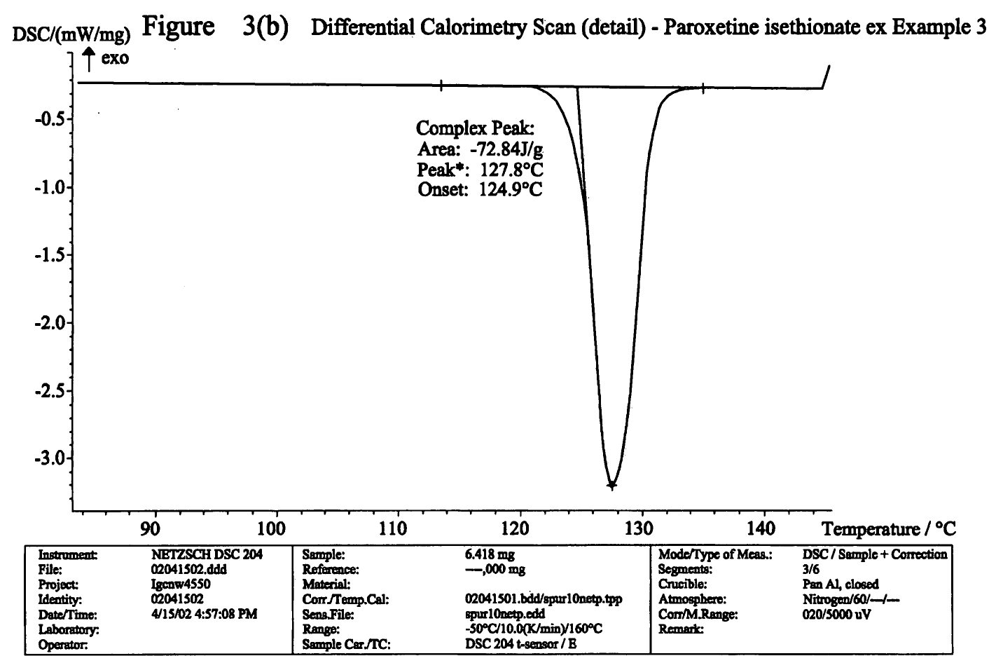

(v) Differential scanning calorimetry: See FIGS. 3(a) and 3(b)

(vi) Solubility in water >1000 mg/ml

### Example 4

Paroxetine free base (1.0 gram) was taken up in methanol (10 ml). Ammonium isethionate (0.45 gram) was then added and the mixture stirred and heated to 60° C. for ten minutes during which time ammonia was evolved. Ethyl acetate (20.0 ml) was added and the mixture distilled to remove methanol with fresh ethyl acetate being added to keep the volume constant. Finally sufficient ethyl acetate was distilled such that the final volume was 20 ml. The solution was allowed to cool with stirring during which time crystals of paroxetine isethionate formed. After cooling to 5° C. the white crystalline product was filtered, washed with ethyl acetate and dried in vacuo.

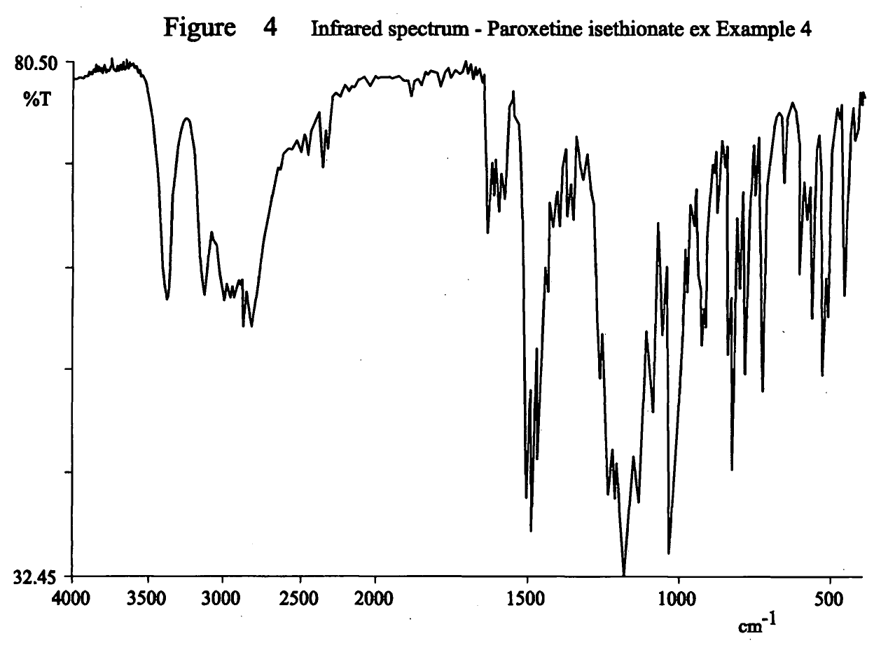

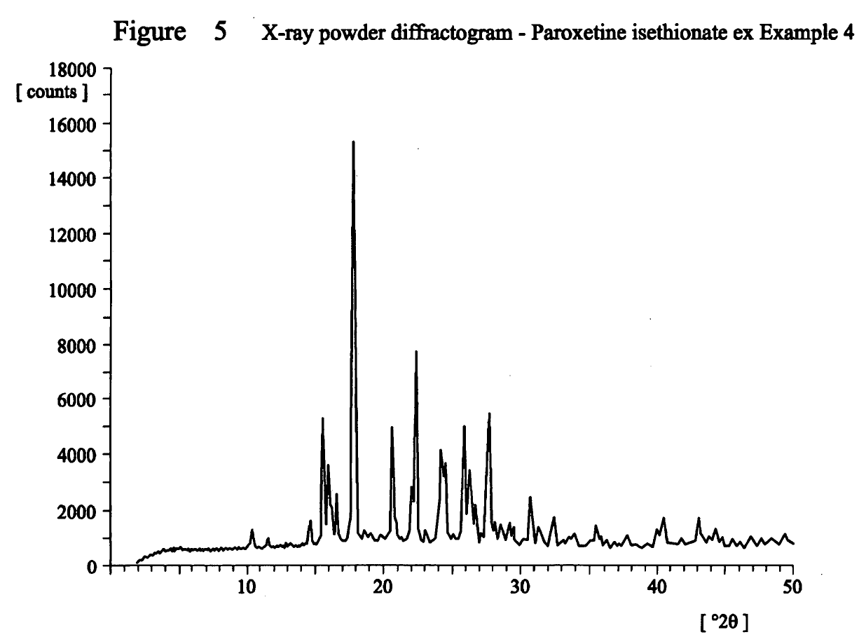

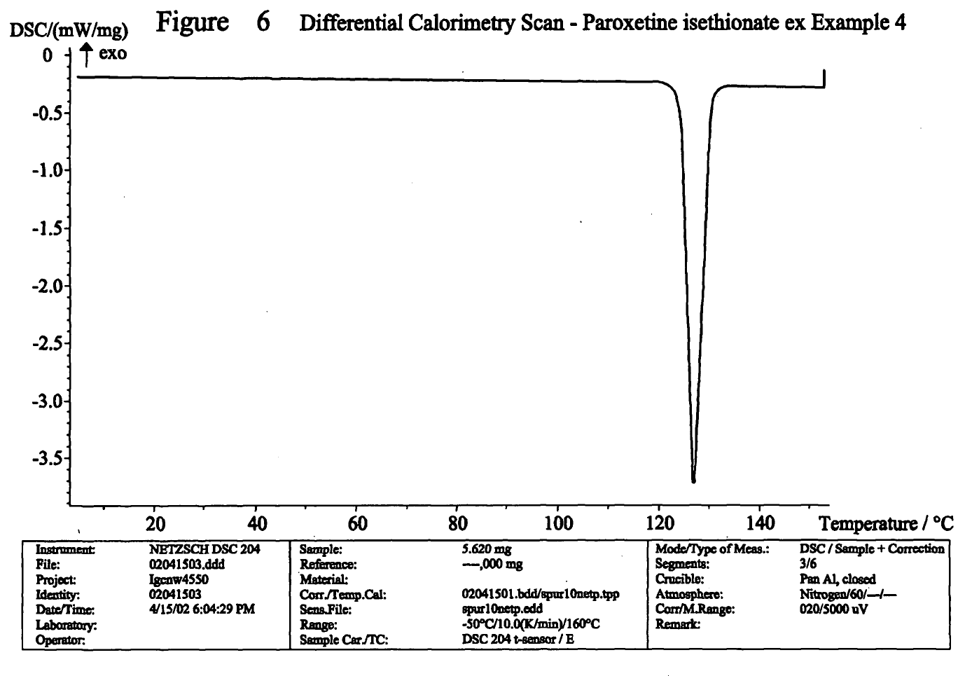

Analytical investigation of this product gave the following results:

| (i) | Elemental analysis | | | |
|---|---|---|---|---|
| | % m/m | C | H | N |
| | Sample | 55.3 | 5.64 | 3.04 |
| | C₂₁H₂₆FNO₇S requires | 55.4 | 5.75 | 3.07 |

| | | |
|---|---|---|
| (ii) | Melting point: | 126.5° C. |
| (iii) | Infrared spectrum: | See FIG. 4 |
| (iv) | X-ray powder diffractogram: | See FIG. 5 |
| (v) | Differential scanning calorimetry: | See FIG. 6 |

### Example 5

Crystalline paroxetine isethionate (1.0 gram) was suspended with stirring in toluene (50 ml). On heating almost to boiling a clear solution was obtained. This solution was allowed to cool with continued stirring when crystals of paroxetine isethionate formed. The mixture was finally cooled to 5° C. and stirred out for two hours. The white crystalline product was filtered, washed with petroleum ether and dried in vacuo.

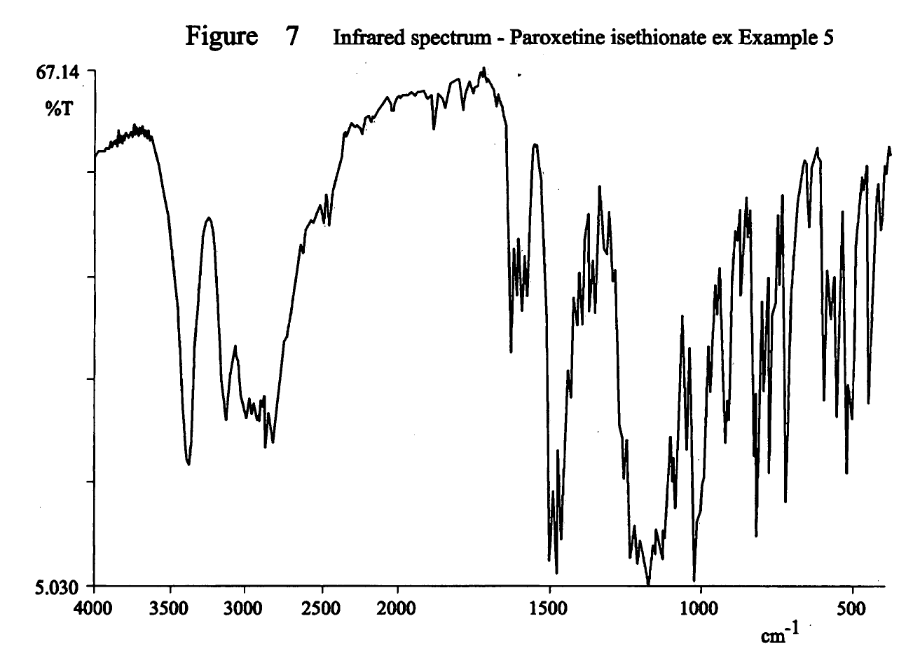

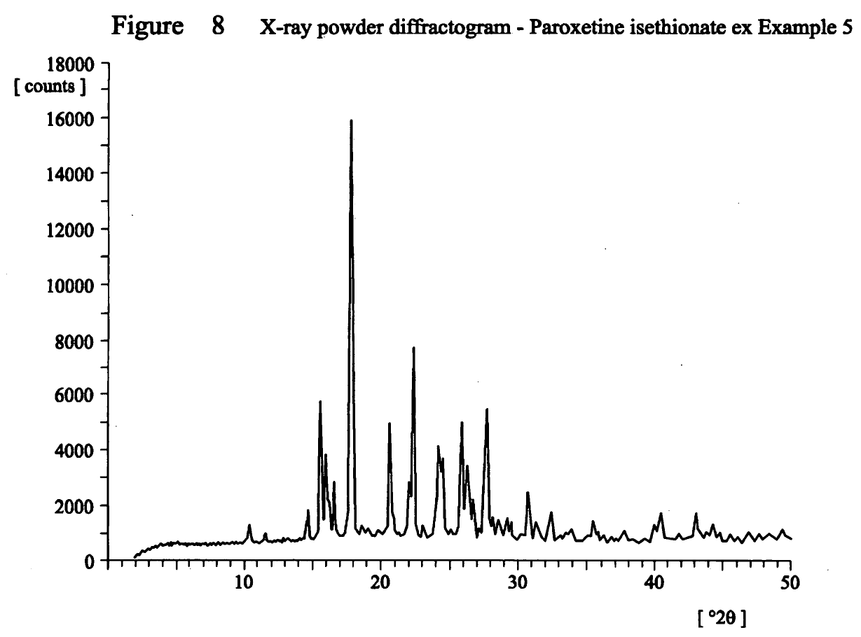

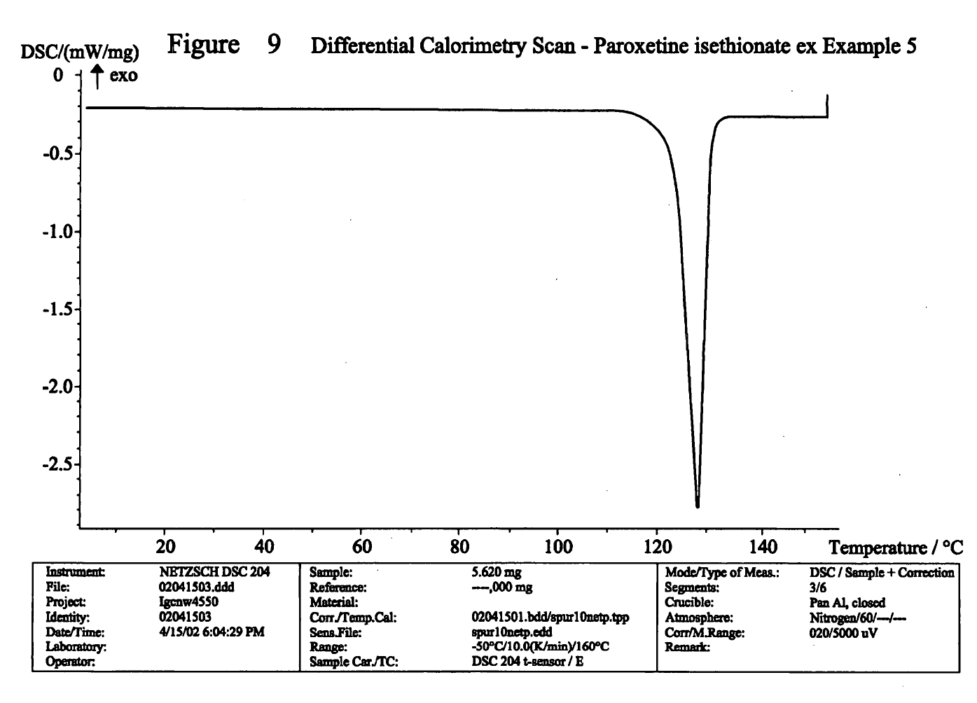

Analytical investigation of this product gave the following results:

| (i) | Elemental analysis | | | |
|---|---|---|---|---|
| | % m/m | C | H | N |
| | Sample | 55.2 | 5.61 | 3.01 |
| | C₂₁H₂₆FNO₇S requires | 55.4 | 5.75 | 3.07 |

| | | |
|---|---|---|
| (ii) | Melting point: | 126.5° C. |
| (iii) | Infrared spectrum: | See FIG. 7 |
| (iv) | X-ray powder diffractogram: | See FIG. 8 |
| (v) | Differential scanning calorimetry: | See FIG. 9 |

### Example 6

An aqueous solution of isethionic acid was prepared from sodium isethionate according to the method described in U.S. Pat. No. 3,812,177. Assay 3.2 molar.

Paroxetine free base (1.0 gram) was taken up in isopropanol (20.0 ml). Aqueous isethionic acid solution 3.2M (1.0 ml) was added with stirring. The mixture was then heated to reflux and isopropanol distilled out keeping the volume constant by the addition of fresh isopropanol until all water had been removed. The volume of the solution was then reduced to 10.0 ml by distillation. On cooling paroxetine isethionate crystallised. The white crystalline product was filtered, washed successively with isopropanol and diethyl ether and dried in vacuo.

### Example 7

Paroxetine isethionate (made according to the method of Example 3) 1.0 gram was dissolved in water 1.0 ml to give a clear quite viscous solution. With good mixing acetone was slowly added in an attempt to crystallise paroxetine isethionate. After the addition of acetone 60 ml no crystals were observed. The solution was chilled to −20° C. and a seed crystal of paroxetine isethionate was added. After standing at −20° C. overnight large crystals of paroxetine isethionate were observed to have been formed. The crystals were recovered by filtration and dried.

With warming to 45° C. paroxetine isethionate 1.0 gram was dissolved in acetone 10 ml with added 1% v/v water. The solution was allowed to cool slowly undisturbed. The large crystals which formed were recovered by filtration and dried.

A solution of paroxetine isethionate 1.0 gram in water 2.0 ml was allowed to evaporate under reduced pressure over silica gel for several days until a clear glass like residue remained. A few crystals of paroxetine isethionate were added and the material left open to the atmosphere. After four days a thick mass of crystalline product was obtained.

### Example 8

Paroxetine acetate (600 mg) was dissolved in isopropanol (10.0 ml). Aqueous isethionic acid solution 3.2M (0.5 ml) was added with stirring. Water was then removed by azeotropic distillation as described in Example 6 above and the final volume reduced to 5.0 ml by distillation. On cooling paroxetine isethionate crystallised. The white crystalline product was filtered, washed successively with isopropanol and diethyl ether and dried in vacuo.

### Example 9

Paroxetine isethionate (1.10 gram) was dissolved in water (4.0 ml). This solution was added to a stirred solution of dilute hydrochloric acid (0.10M, 25 ml) at 40° C. over a period of 15 minutes. The resulting white suspension was cooled to 20° C. with continued stirring and the product filtered. After washing with a little water the white crystalline paroxetine hydrochloride hemi hydrate was dried in vacuo.

### Example 10

N-tertiary butyloxycarbonyl paroxetine (0.33 gram) was dissolved in isopropanol and aqueous isethionic acid solution 3.2M (0.25 ml) was added. The mixture was left to stand at room temperature for 96 hours. Solvent and water were then removed by heating at 60° C. under reduced pressure. The oily residue was taken up in hot isopropanol (5.0 ml) and the solvent again removed at 60° C. to remove last traces of water. The residue was triturated with a little ethyl acetate in the presence of a few seed crystals of paroxetine isethionate. The resulting crystalline paroxetine isethionate was slurried in additional ethyl acetate, filtered and dried in vacuo.

### Example 11

For tablets containing paroxetine 30 mg (as free base) Ingredients:

| | |
|---|---|
| Paroxetine isethionate (100%) | 41.5 mg |
| Dicalcium phosphate | 125.0 mg |
| Microcrystalline cellulose | 76.0 mg |
| Sodium starch glycollate | 12.5 mg |
| Magnesium stearate | 2.5 mg |

The required quantity of dicalcium phosphate was sieved and then paroxetine isethionate added. Microcrystalline cellulose and sodium starch glycollate were added and the powders mixed for ten minutes. The magnesium stearate was added and mixed for a further five minutes. The tablets were made with a single punch tablet press.

TABLE 1
X-ray powder diffractogram - Paroxetine isothionate Example 3
Conditions and listing of peak positions and intensities

| Diffractometer type: | PW1800 |
|---|---|
| Tube anode: | Cu |
| Generator tension [kV]: | 40 |
| Generator current [mA]: | 55 |
| Wavelength Alpha1 [Å]: | 1.54056 |
| Wavelength Alpha2 [Å]: | 1.54439 |
| Intensity ratio (alpha2/alpha1): | 0.500 |
| Divergence slit: | FINE |
| Irradiated length [mm]: | 2 |
| Receiving slit: | COARSE |
| Spinner: | OFF |
| Monochromator used: | YES |
| Start angle [2° θ]: | 2.010 |
| End angle [2° θ]: | 69.990 |
| Step size [2° θ]: | 0.020 |
| Maximum intensity: | 149305.0 |
| Time per step [s]: | 4.000 |
| Type of scan: | CONTINUOUS |
| Intensities converted to: | FIXED |
| Peak positions defined by: | Top of smoothed peak |
| Minimum peak tip width: | 0.00 |
| Maximum peak tip width: | 1.00 |
| Peak base width: | 2.00 |
| Minimum significance: | 0.75 |
| Number of peaks: | 78 |

| | | | Peak | | Back. | Rel. | |
|---|---|---|---|---|---|---|---|
| Angle | d-value | d-value | width | Peak int | int | int | |
| [°2θ] | α1 [Å] | α2 [Å] | [°2θ] | [counts] | [counts] | [%] | Signif. |
| 10.365 | 8.5276 | 8.5488 | 0.080 | 10120 | 9624 | 6.8 | 2.24 |
| 11.520 | 7.6750 | 7.6941 | 0.120 | 4556 | 8593 | 3.1 | 2.37 |
| 12.875 | 6.8702 | 6.8873 | 0.200 | 2070 | 7691 | 1.4 | 1.62 |
| 13.225 | 6.6891 | 6.7058 | 0.120 | 2285 | 7430 | 1.5 | 1.32 |
| 14.615 | 6.0559 | 6.0710 | 0.100 | 13248 | 6724 | 8.9 | 4.93 |
| 15.585 | 5.6811 | 5.6952 | 0.100 | 57073 | 6257 | 38.2 | 14.77 |
| 15.965 | 5.5468 | 5.5605 | 0.100 | 36062 | 6100 | 24.2 | 7.60 |
| 16.170 | 5.4769 | 5.4905 | 0.080 | 17004 | 6037 | 11.4 | 2.22 |
| 16.575 | 5.3440 | 5.3572 | 0.100 | 19909 | 5837 | 13.3 | 5.52 |
| 17.830 | 4.9705 | 4.9829 | 0.140 | 149305 | 5432 | 100.0 | 49.25 |
| 18.700 | 4.7412 | 4.7530 | 0.080 | 5314 | 5127 | 3.6 | 1.24 |
| 19.075 | 4.6488 | 4.6604 | 0.080 | 4570 | 5027 | 3.1 | 0.93 |
| 19.820 | 4.4757 | 4.4869 | 0.120 | 3272 | 4802 | 2.2 | 0.75 |
| 20.625 | 4.3028 | 4.3135 | 0.140 | 37520 | 4624 | 25.1 | 20.63 |
| 20.945 | 4.2378 | 4.2483 | 0.100 | 6691 | 4543 | 4.5 | 0.79 |
| 22.080 | 4.0225 | 4.0325 | 0.100 | 16180 | 4277 | 10.8 | 4.20 |
| 22.375 | 3.9701 | 3.9800 | 0.140 | 54103 | 4225 | 36.2 | 31.72 |
| 23.135 | 3.8414 | 3.8509 | 0.080 | 4449 | 4058 | 3.0 | 1.07 |
| 24.185 | 3.6769 | 3.6861 | 0.140 | 24649 | 3881 | 16.5 | 10.20 |
| 24.570 | 3.6202 | 3.6292 | 0.120 | 21228 | 3819 | 14.2 | 7.21 |
| 25.070 | 3.5491 | 3.5579 | 0.060 | 3364 | 3709 | 2.3 | 1.77 |
| 25.945 | 3.4314 | 3.4399 | 0.140 | 27989 | 3588 | 18.7 | 17.29 |
| 26.335 | 3.3814 | 3.3898 | 0.140 | 17929 | 3505 | 12.0 | 14.23 |
| 26.675 | 3.3391 | 3.3474 | 0.180 | 12166 | 3457 | 8.1 | 15.78 |
| 27.155 | 3.2811 | 3.2893 | 0.100 | 3260 | 3399 | 2.2 | 1.08 |
| 27.650 | 3.2235 | 3.2315 | 0.140 | 28696 | 3341 | 19.2 | 13.79 |
| 27.800 | 3.2065 | 3.2144 | 0.100 | 21083 | 3329 | 14.1 | 4.27 |
| 28.110 | 3.1718 | 3.1797 | 0.060 | 5055 | 3260 | 3.4 | 2.69 |
| 28.500 | 3.1293 | 3.1370 | 0.080 | 5013 | 3215 | 3.4 | 0.83 |
| 28.700 | 3.1079 | 3.1156 | 0.100 | 4343 | 3192 | 2.9 | 0.85 |
| 29.165 | 3.0594 | 3.0670 | 0.120 | 6100 | 3147 | 4.1 | 5.29 |
| 29.465 | 3.0289 | 3.0365 | 0.160 | 4290 | 3114 | 2.9 | 6.40 |
| 30.300 | 2.9474 | 2.9547 | 0.080 | 2125 | 3003 | 1.4 | 0.76 |
| 30.740 | 2.9062 | 2.9134 | 0.160 | 9761 | 2959 | 6.5 | 15.03 |
| 31.350 | 2.8510 | 2.8581 | 0.100 | 4070 | 2884 | 2.7 | 1.64 |
| 32.405 | 2.7605 | 2.7674 | 0.180 | 5791 | 2788 | 3.9 | 11.78 |
| 33.000 | 2.7121 | 2.7188 | 0.240 | 1399 | 2714 | 0.9 | 1.13 |
| 33.525 | 2.6708 | 2.6775 | 0.120 | 1772 | 2673 | 1.2 | 1.55 |
| 34.030 | 2.6323 | 2.6389 | 0.120 | 2540 | 2632 | 1.7 | 4.23 |
| 35.460 | 2.5294 | 2.5357 | 0.120 | 4290 | 2510 | 2.9 | 4.09 |
| 35.800 | 2.5061 | 2.5124 | 0.100 | 1849 | 2490 | 1.2 | 0.86 |
| 36.095 | 2.4863 | 2.4925 | 0.060 | 1444 | 2440 | 1.0 | 0.80 |
| 36.355 | 2.4692 | 2.4753 | 0.060 | 1303 | 2430 | 0.9 | 0.90 |
| 37.240 | 2.4125 | 2.4185 | 0.160 | 912 | 2372 | 0.6 | 1.07 |
| 37.815 | 2.3771 | 2.3830 | 0.200 | 2343 | 2314 | 1.6 | 3.31 |
| 38.420 | 2.3411 | 2.3469 | 0.160 | 847 | 2285 | 0.6 | 0.82 |
| 39.355 | 2.2876 | 2.2932 | 0.160 | 955 | 2209 | 0.6 | 0.93 |
| 40.020 | 2.2511 | 2.2567 | 0.160 | 2873 | 2172 | 1.9 | 3.58 |
| 40.400 | 2.2308 | 2.2363 | 0.140 | 4872 | 2153 | 3.3 | 5.28 |
| 40.495 | 2.2258 | 2.2313 | 0.060 | 3624 | 2153 | 2.4 | 0.90 |
| 40.820 | 2.2088 | 2.2143 | 0.160 | 1225 | 2116 | 0.8 | 0.79 |
| 41.685 | 2.1649 | 2.1703 | 0.120 | 1253 | 2070 | 0.8 | 1.20 |
| 41.890 | 2.1548 | 2.1602 | 0.120 | 1232 | 2061 | 0.8 | 1.26 |
| 42.490 | 2.1258 | 2.1310 | 0.120 | 1043 | 2016 | 0.7 | 0.91 |
| 42.950 | 2.1040 | 2.1093 | 0.180 | 4733 | 1998 | 3.2 | 8.36 |
| 43.810 | 2.0647 | 2.0698 | 0.160 | 1600 | 1945 | 1.1 | 1.17 |
| 44.340 | 2.0413 | 2.0463 | 0.080 | 3091 | 1918 | 2.1 | 1.24 |
| 44.780 | 2.0222 | 2.0272 | 0.140 | 1866 | 1901 | 1.2 | 1.96 |
| 45.515 | 1.9913 | 1.9962 | 0.160 | 1050 | 1858 | 0.7 | 1.11 |
| 46.025 | 1.9704 | 1.9753 | 0.240 | 858 | 1832 | 0.6 | 2.31 |
| 46.875 | 1.9366 | 1.9414 | 0.120 | 1429 | 1789 | 1.0 | 1.17 |
| 47.610 | 1.9084 | 1.9131 | 0.120 | 1296 | 1764 | 0.9 | 1.55 |
| 48.350 | 1.8809 | 1.8856 | 0.120 | 1050 | 1739 | 0.7 | 0.80 |
| 48.565 | 1.8731 | 1.8777 | 0.160 | 1136 | 1714 | 0.8 | 0.88 |
| 49.360 | 1.8448 | 1.8493 | 0.240 | 1866 | 1689 | 1.2 | 4.39 |

1. A salt comprising an anion of isethionic acid and a cation derived from paroxetine free base of formula (I)

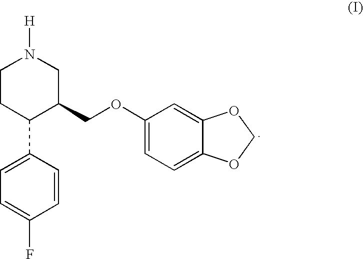

2. Paroxetine isethionate of formula (II) 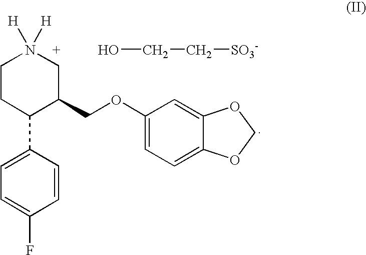
.

3. (cancelled).

4. A process of preparing a paroxetine salt which process comprises reacting isethionic acid, or a salt thereof, and paroxetine free base of formula (I) 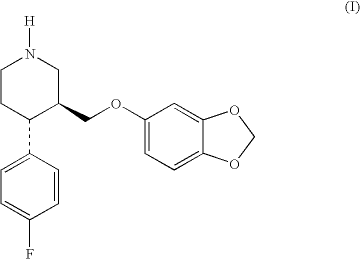
or a salt thereof, in a solvent and isolating the resulting salt of paroxetine.

5. A process according to claim 4, which comprises reacting paroxetine free base of formula (I) with the ammonium salt of isethionic acid.

6. A process according to claim 4, wherein the solvent comprises one or more of the following:
water;
C₁-C₆ alkanols;
C₁-C₆ ketones;
acetonitrile; and
dimethylformamide.

7. A process according to claim 4, which further comprises converting the resulting salt of paroxetine into a further pharmaceutically acceptable salt.

8. A pharmaceutical composition comprising a salt according claim 1, together with a pharmaceutically acceptable carrier, diluent or excipient therefor.

9-11. (cancelled)

12. A method of treatment of an animal patient suffering from, or susceptible to, a disease state ameliorated by the administration of a 5HT uptake inhibitor, which method comprises administering an effective amount of a salt according claim 1 to the animal patient.

13. A method according to claim 12, wherein the disease state is depression or anxiety disorder.

14. (cancelled)

15. (cancelled)

16. A pharmaceutical composition comprising a salt according claim 2, together with a pharmaceutically acceptable carrier, diluent or excipient therefor.

17. A method of treatment of an animal patient suffering from, or susceptible to, a disease state ameliorated by the administration of a 5HT uptake inhibitor, which method comprises administering an effective amount of a salt according claim 2 to the animal patient.

18. A method according to claim 17, wherein the disease state is depression or anxiety disorder.

19. A process according to claim 5, wherein the solvent comprises one or more of the following:
water;
C₁-C₆ alkanols;
C₁-C₆ ketones;
acetonitrile; and
dimethylformamide.

20. A method of treatment of an animal patient suffering from, or susceptible to, a disease state ameliorated by the administration of a 5HT uptake inhibitor, which method comprises administering an effective amount of a salt according claim 2 to the animal patient.

21. A method according to claim 20, wherein the disease state is depression or anxiety disorder.

22. The process of claim 4, wherein the is selected from among solvents that promote ionisation.

23. The process of claim 22, wherein seed crystals are present during the reaction of the isethionic acid, or a salt thereof, and paroxetine free base, or salt thereof.

24. The process of claim 4, wherein seed crystals are present during the reaction of the isethionic acid, or a salt thereof, and paroxetine free base, or salt thereof.

25. A salt comprising an anion of isethionic acid and a cation derived from paroxetine free base of formula (I) 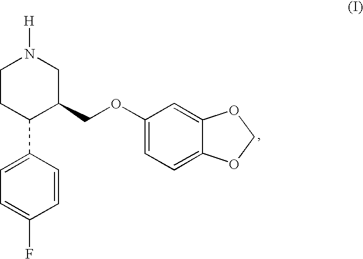
wherein the salt is in a crystalline form.

26. The salt of claim 25, wherein the salt is a hydrate or solvate.

27. The salt of claim 25, wherein the salt is paroxetine isethionate of formula (II) [chemical structure]
